(19) United States
(12) Patent Application Publication
(10) Pub. No.: US 2023/0329605 A1
(43) Pub. Date: Oct. 19, 2023

(54) Blood Draw Device with Catheter Length Adjustable Advancement Stop and Tube Position Indicator

(71) Applicant: Becton, Dickinson and Company, Franklin Lakes, NJ (US)

(72) Inventors: Jonathan Karl Burkholz, Salt Lake City, UT (US); Megan S. Scherich, Salt Lake City, UT (US); Weston F. Harding, Lehi, UT (US); Yiping Ma, Layton, UT (US); Ralph L. Sonderegger, Farmington, UT (US)

(21) Appl. No.: 18/135,826

(22) Filed: Apr. 18, 2023

Related U.S. Application Data
(60) Provisional application No. 63/332,484, filed on Apr. 19, 2022.

(51) Int. Cl. A61B 5/15

(52) U.S. Cl. CPC A61B 5/15019; A61B 5/150992; A61B 5/150816

(57) ABSTRACT

A blood draw device for delivery of a probe into a patient's vascular system, the blood draw device including an introducer body having a proximal end and a distal end, and an actuator. The actuator is displaceable along at least a portion of the introducer body, and the actuator is operably coupled to the probe to advance and retract the probe based on direction of displacement of the actuator. The blood draw device also includes an advancement stop member, wherein the advancement stop member is displaceable along at least a portion of the introducer body and is configured to selectively limit travel of the actuator along the introducer body.

### CROSS-REFERENCE TO RELATED APPLICATION

The present application claims priority to U.S. Provisional Application Ser. No. 63/332,484, entitled “Blood Draw Device with Catheter Length Adjustable Advancement Stop and Tube Position Indicator”, filed Apr. 19, 2022, the entire disclosure of which is hereby incorporated by reference in its entirety.

### BACKGROUND OF THE INVENTION

### Field of the Invention

The present disclosure generally relates to blood draw devices and related assemblies, systems, and methods for use with catheter such as, e.g., peripheral intravenous catheters (PIVCs). More particularly, the blood draw devices are configured to include an advancement stop and tube (or “probe”) position indicator adjustable for indwelling catheters of various lengths.

### Description of Related Art

A catheter is commonly used to infuse fluids into vasculature of a patient. For example, the catheter may be used for infusing normal saline solution, various medicaments, or total parenteral nutrition. Furthermore, the catheter may also be used for withdrawing blood from the patient.

The catheter may be an over-the-needle peripheral intravenous catheter (PIVC). In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. After proper placement of the needle, the clinician may temporarily occlude flow in the vasculature and remove the needle, leaving the catheter in place (i.e., “indwelled”) for future blood withdrawal and/or fluid infusion.

In order to complete blood draws from PIVCs having indwelled catheters, blood draw devices have been developed that are configured to overcome previous challenges related to blood draw through PIVCs such as, e.g., the possibility of catheter collapse, reduced blood flow due to debris built up on or within the catheter, etc. One such device, PIVO™ from Velano Vascular, Inc., is configured as a single-use device which temporarily attaches to a PIVC to draw a blood sample. Using an existing peripheral intravenous line as a conduit to the vasculature, the PIVO™ device advances a flexible, internal flow tube through the PIVC, beyond the catheter tip, and into the vein to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded. An example of one such blood draw device is shown and described in U.S. Pat. No. 10,300,247 B2, which is incorporated by reference herein in its entirety.

However, in some instances, PIVCs are offered with indwelling catheters of varying lengths, with the specific length being selected based on, e.g., patient anatomy, application, etc. For example, the BD NEXIVA™ Closed IV Catheter System from Becton Dickinson, and Company provides 20 gauge PIVCs with catheter lengths of 1.00 in., 1.25 in., and 1.75 in., respectively. While these catheters having various lengths may allow for improved placement of the PIVC into a patient's vasculature, they may prove problematic when utilized with a blood draw device configured for blood collection via the PIVC. Specifically, the flow tube of the blood draw device typically has a maximum extension length to which it can be advanced beyond the indwelling catheter's distal tip. While that maximum length of the flow tube may be sufficient for suitable protrusion from a PIVC having a catheter length of, e.g., 1.00 in., it may not provide adequate protrusion from a PIVC having a catheter length of, e.g., 1.75 in. Conversely, if the fluid transfer device were designed to provide suitable protrusion from a PIVC having a longer catheter length (e.g., 1.75 in.), the flow tube of that same device may extend an undesirable distance beyond the tip of a shorter (e.g., 1.00 in.) catheter, and the user may receive no indication as to how far the flow tube extends beyond the distal tip of the catheter.

### SUMMARY OF THE INVENTION

Accordingly, the present disclosure generally relates to blood draw devices used for blood draw via indwelling catheters and related assemblies, systems, and methods. The blood draw devices are provided with an advancement stop member selectively positioned by a user such that the length of a flexible probe extending from the fluid transfer device is compatible with indwelling catheters of varying length. Additionally, a proximal portion of the advancement stop member may be provided with indicators to specify a position of the distal end of the probe relative to a catheter tip.

In accordance with an embodiment of the present disclosure, a blood draw device for delivery of a probe into a patient's vascular system is disclosed. The blood draw device may include an introducer body having a proximal end and a distal end, and an actuator, wherein the actuator is displaceable along at least a portion of the introducer body, and wherein the actuator is operably coupled to the probe to advance and retract the probe based on direction of displacement of the actuator. The blood draw device may also include an advancement stop member, wherein the advancement stop member is displaceable along at least a portion of the introducer body and is configured to selectively limit travel of the actuator along the introducer body.

In some embodiments, the advancement stop member may include a distal interface portion and a probe position indicator portion, wherein the probe position indicator portion is coupled to and extends proximally from the distal interface portion.

In some embodiments, the probe position indicator portion extends along at least one sidewall of the introducer body.

In some embodiments, the probe position indicator portion extends along two opposing sides of the introducer body.

In some embodiments, probe position indicator portion includes a plurality of probe position indicators, wherein each of the probe position indicators relate to a position of a distal tip of the probe relative to a distal tip of an indwelling catheter to which the blood draw device is coupled.

In some embodiments, the plurality of probe position indicators include visual indicators.

In some embodiments, the visual indicators include indications of when the distal tip of the probe is a) at the distal tip of the indwelling catheter, b) 1 cm beyond the distal tip of the indwelling catheter, c) 2 cm beyond the distal tip of the indwelling catheter, and d) 3 cm beyond the distal tip of the indwelling catheter.

In some embodiments, the plurality of probe position indicators are configured to provide tactile feedback to the user.

In some embodiments, the introducer body includes a plurality of catheter length indicators formed on a distal end portion thereof.

In some embodiments, each of the catheter length indicators corresponds to a length of a catheter to which the blood draw device is coupled.

In some embodiments, the advancement stop member is configured to selectively engage with the catheter length indicators so as to provide discrete stop positions for the advancement stop member along the introducer body.

In some embodiments, each of the catheter length indicators include one of a projection or a detent.

In some embodiments, each of the catheter length indicators include visual indicia pertaining to a catheter length.

In some embodiments, the blood draw device further includes a connector and a blunt introducer extending from the distal end of the introducer body.

In accordance with another aspect of the present disclosure, a blood draw device for delivery of a probe into a patient's vascular system is disclosed. The blood draw device may include an introducer body having a proximal end and a distal end, and an actuator, wherein the actuator is displaceable along at least a portion of the introducer body, and wherein the actuator is operably coupled to the probe to advance and retract the probe based on direction of displacement of the actuator. The blood draw device may also include an advancement stop member, wherein the advancement stop member is rotatable about the introducer body and is configured to selectively limit travel of the actuator along the introducer body.

In some embodiments, the advancement stop member may include a proximally-directed stepped portion configured to extend into the introducer body.

According to another aspect of the present disclosure, a method of blood collection from a vascular access device is disclosed. The method may include providing a catheter assembly having a catheter of known length, and providing a blood draw device, the blood draw device including an introducer body having a proximal end and a distal end, an actuator, wherein the actuator is displaceable along at least a portion of the introducer body, and wherein the actuator is operably coupled to a flexible probe to advance and retract the probe based on direction of displacement of the actuator, and an advancement stop member, wherein the advancement stop member is displaceable along at least a portion of the introducer body and is configured to selectively limit travel of the actuator along the introducer body. The method may further include adjusting the advancement stop member to a position on the introducer body based on the known length of the catheter, coupling the blood draw device to the catheter assembly, advancing the actuator to correspondingly advance the probe from the blood draw device and beyond a distal tip of the catheter, and collecting a blood sample via the blood draw device.

In some embodiments, the advancement stop member includes a distal interface portion and a probe position indicator portion, and the probe position indicator portion is coupled to and extends proximally from the distal interface portion.

In some embodiments, the step of advancing the actuator on the blood draw device may include using the probe position indicator portion as a guide to determine desired travel of the probe beyond the distal tip of the catheter.

In some embodiments, the method may further include retracting the probe from the catheter after collection of the blood sample using the actuator, and decoupling the blood draw device from the catheter assembly.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a perspective view of a catheter assembly and blood draw device in accordance with an aspect of the present disclosure;

FIG. 2 is a partial perspective view of the blood draw device of FIG. 1;

FIG. 3A is a rear perspective view of a blood draw device in accordance with another aspect of the present disclosure in a first configuration;

FIG. 3B is a rear perspective view of the blood draw device of FIG. 3A in a second configuration;

FIG. 4 is a side view of the blood draw device of FIGS. 3A and 3B in the second configuration;

FIG. 5 is a partial cross-sectional view of a blood draw device in accordance with another aspect of the present disclosure;

FIG. 6 is a partial top plan view of a blood draw device in accordance with another aspect of the present disclosure;

FIG. 7A is a perspective view of a blood draw device in accordance with another aspect of the present disclosure;

FIG. 7B is a partial cross-sectional view of the blood draw device of FIG. 7A in a first configuration;

FIG. 7C is a partial cross-sectional view of the blood draw device of FIG. 7A in a second configuration;

FIG. 8A is a perspective view of a blood draw device in accordance with another aspect of the present disclosure;

FIG. 8B is a partial cross-sectional view of the blood draw device of FIG. 8A in a first configuration;

FIG. 8C is a partial cross-sectional view of the blood draw device of FIG. 8A in a second configuration;

FIG. 9A is a perspective view of a blood draw device in accordance with another aspect of the present disclosure;

FIG. 9B is a partial cross-sectional view of the blood draw device of FIG. 9A in a first configuration;

FIG. 9C is a partial cross-sectional view of the blood draw device of FIG. 9A in a second configuration;

FIG. 10A is a partial cross-sectional view of a blood draw device in accordance with another aspect of the present disclosure in a first configuration;

FIG. 10B is a partial cross-sectional view of the blood draw device of FIG. 10A in a second configuration;

FIG. 10C is a partial cross-sectional view of the blood draw device of FIG. 10A in a third configuration;

FIG. 11 is a partial top plan view of a blood draw device in accordance with another aspect of the present disclosure;

FIG. 12 is a partial top plan view of a blood draw device in accordance with another aspect of the present disclosure; and

FIG. 13 illustrates method steps of using a blood draw device in accordance with an aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a fluid transfer device in preparation for or during use. Similarly, in this application, the terms “in the distal direction” and “distally” mean in the direction toward the connector portion of the fluid transfer device, and the terms “in the proximal direction” and “proximally” mean in the direction opposite the direction of the connector.

While not shown or described herein, it is to be understood that the blood draw devices described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood draw devices for use with PIVCs. However, embodiments of the present disclosure equally extend to use with other catheter devices.

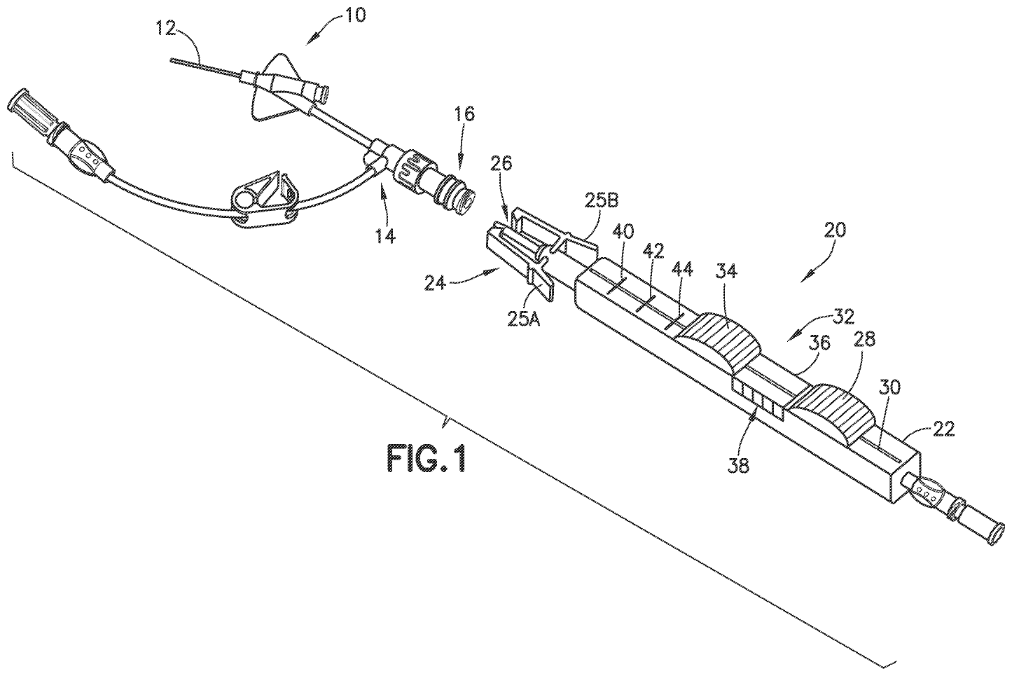

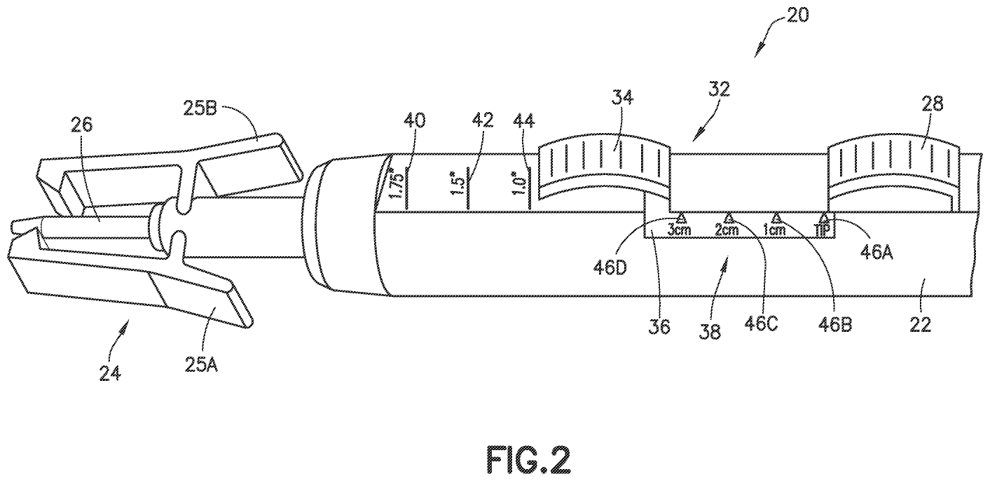

Referring to FIGS. 1 and 2, a blood draw device 20 in accordance with an aspect of the present disclosure is illustrated. As shown in FIG. 1, blood draw device 20 is configured to be releasably couplable to an adapter 14 having a needleless access connector 16, with the adapter 14 coupled to a catheter adapter 10 having a catheter 12 extending therefrom and configured for vascular access. The blood draw device 20 includes an introducer body 22 having a connector 24 and a blunt introducer 26 positioned at a distal end portion. While not shown, it is to be understood that a proximal end of the blood draw device 20 may be fluidly coupled to a blood collection container such as, e.g., a BD VACUTAINER® blood collection tube in order to collect a blood sample from the patient via the vascular access device.

While not shown, the blood draw device 20 includes an elongated tube or probe (e.g., a nickel titanium wire, guidewire, instrument, obturator, rod, wire with fluid path and/or sensor, etc.) that can be selectively advanced in a distal direction through the adapter 14, the catheter adapter 10, and beyond the distal tip of the catheter 12, with the probe providing a fluid channel to allow for blood draw into a blood collection device coupled to the blood draw device 20. In the embodiment shown in FIGS. 1 and 2, advancement of the probe of blood draw device 20 is controlled by an actuator 28 operably coupled to the probe and linearly slidable along a slot 30 formed in or on the introducer body 22, which enables a clinician to use his or her thumb to push the actuator 28 in a distal direction to advance the probe and, conversely, in a proximal direction to retract the probe. However, it is to be understood that actuator 28 may include any surface or feature that is operatively coupled to any point of the probe that the user interacts with to advance and retracted the fluid path or probe. Accordingly, actuator 28 is not limited to an actuator capable of linearly sliding along a slot formed in the introducer body, and may be any feature capable of advancing and retracting the probe.

Prior to deployment of the probe, the blood draw device 20 is coupled to the adapter 14, with the blunt introducer 26 being inserted through a septum in the needleless access connector 16. Upon sufficient distal insertion of the blunt introducer 26 within the needleless access connector 16, the releasable proximal clip portions 25A, 25B of distal connector 24 are configured to clip onto the adapter 14 to secure the blood draw device 20 in place. Once secured, the probe may be advanced from the blood draw device by way of linear displacement of the actuator 28.

As noted above, the indwelling catheter 12 may have various lengths and/or gauges, with the specific length and/or gauge being selected based on, e.g., patient anatomy, application, location, etc. For example, the catheter 12 may have lengths of 0.5 inches, 0.75 inches, 1.0 inch, 1.25 inches, 1.5 inches, or 1.75 inches. However, it is to be understood that these lengths are not limiting, and the catheter 12 may be longer or shorter. Additionally, catheter 12 may have varying gauges such as, e.g., 18 G, 20 G, 22 G, or 24 G. However, as with catheter length, the catheter 12 may have any appropriate gauge and is not limited to the above examples.

Referring still to FIGS. 1 and 2, the blood draw device 20 further includes an advancement stop member 32. Similar to actuator 28, advancement stop member 32 is linearly displaceable along the introducer body 22 of blood draw device 20. In some embodiments, the advancement stop member 32 may have a slight interference fit with the introducer body 22, thereby avoiding unintentional movement or displacement of the advancement stop member 32.

In some embodiments, the advancement stop member 32 includes a distal interface portion 34, which provides the user with an interface for selectively advancing or retracting the advancement stop member 32. In some embodiments, an upper surface of the distal interface portion 34 may include ribs or other textured surface features so as to improve the user's ability to manipulate the advancement stop member 32 along the introducer body 22.

Upon an upper or external surface of the introducer body 22 near a distal end portion of the blood draw device 20, a plurality of catheter length indicators 40, 42, 44 are provided. The catheter length indicators 40, 42, 44 may be projections or detents formed on or in the introducer body 22 so as to provide discrete stop positions for the advancement stop member 32. In some embodiments, the projections or detents of the catheter length indicators 40, 42, 44 may interact with a downward-projecting tab on the distal interface portion 34 so as to selectively engage the advancement stop member 32, with the user needing to press downward or otherwise manipulate the distal interface portion 34 when movement of the advancement stop member 32 to another catheter length indicator is desired. Each of the catheter length indicators 40, 42, 44 refer to a catheter length usable in conjunction with the blood draw device 20. In the example shown in FIG. 2, catheter length indicator 40 demarcates a position for a 1.75 inch catheter, catheter length indicator 42 demarcates a position for a 1.5 inch catheter, and catheter length indicator 44 demarcates a position for a 1.00 inch catheter. However, it is to be understood that the blood draw device 20 is not limited to these catheter lengths, and more or fewer catheter length indicators may be provided on the introducer body 22.

Prior to the user distally advancing the probe from the blood draw device 20 via advancement of the actuator 28, the user first sets the advancement stop member 32 at an appropriate position relative to a known catheter length. For example, in the embodiment shown in FIGS. 1 and 2, the advancement stop member 32 is positioned at catheter length indicator 44, which relates to a 1.0 inch catheter length. While not shown, the catheter adapter 10 and/or catheter 12 may include a visual indicator as to catheter length and/or gauge, thereby ensuring that the user is able to position the advancement stop member 32 at an appropriate catheter length indicator on the blood draw device 20. Depending on the position of advancement stop member 32, linear travel of the actuator 28 along the introducer body 22 is limited, thereby also limiting the linear advancement of the probe from the blood draw device 20. That is, for a shorter catheter (e.g., a 1.0 inch catheter, as shown in FIGS. 1 and 2), linear travel of actuator 28 is limited due to the distal interface portion 34 forming a physical stop for actuator 28 at a distance proximally away from a distal end of the introducer body 22. However, if a longer catheter is used (e.g., a 1.75 inch catheter), the advancement stop member 32 is to be moved distally to the catheter length indicator 40, which allows for greater linear travel of actuator 28 and, thus, increased advancement of the probe relative to the distal end of the blood draw device 20, thereby accounting for the increased catheter length.

Referring again to FIGS. 1 and 2, in some embodiments, the advancement stop member 32 further includes a probe position indicator portion 38. The probe position indicator portion 38 is coupled to, and extends proximally from, the distal interface portion 34, and also extends laterally along at least one side of the introducer body 22. The probe position indicator portion 38 is configured to allow substantially unencumbered linear movement of the actuator 28 therethrough, but provides probe position indicators 46A-46D on a side surface 36 thereof. In some embodiments, the probe position indicators 46A-46D are indicators related to the position of the distal tip of the probe beyond the distal tip of the catheter 12 based on the position of the actuator 28. For example, as shown in FIG. 2, the probe position indicator 46A provides an indication as to when the distal tip of the probe is positioned at (or substantially near) the distal tip of the catheter 12, the probe position indicator 46B provides an indication as to when the distal tip of the probe is positioned approximately 1 cm beyond the distal tip of the catheter 12, the probe position indicator 46C provides an indication as to when the distal tip of the probe is positioned approximately 2 cm beyond the distal tip of the catheter 12, and the probe position indicator 46D provides an indication as to when the distal tip of the probe is positioned approximately 3 cm beyond the distal tip of the catheter 12.

In this way, the user is able to verify the advanced position of the probe beyond the distal tip of the indwelling catheter 12, helping to avoid overextension (or underextension) of the probe beyond the distal tip of the indwelling catheter 12. Furthermore, because the probe position indicator portion 38 is coupled to the distal interface portion 34 of the advancement stop member 32, the distal advancement of the probe is able to be accurately determined and monitored, regardless of the length of the catheter 12.

In some embodiments, the probe position indicators 46A-46D are solely visual indicators, providing the user with a visual confirmation as to the advancement distance of the probe beyond the catheter 12. However, in other embodiments, the probe position indicators 46A-46D may provide tactile and/or audible feedback to the user related to the various distal positions of the probe. Additionally and/or alternatively, while four probe position indicators 46A-46D are shown and described with respect to FIGS. 1 and 2, it is to be understood that more or fewer probe position indicators may be utilized in accordance with the present disclosure.

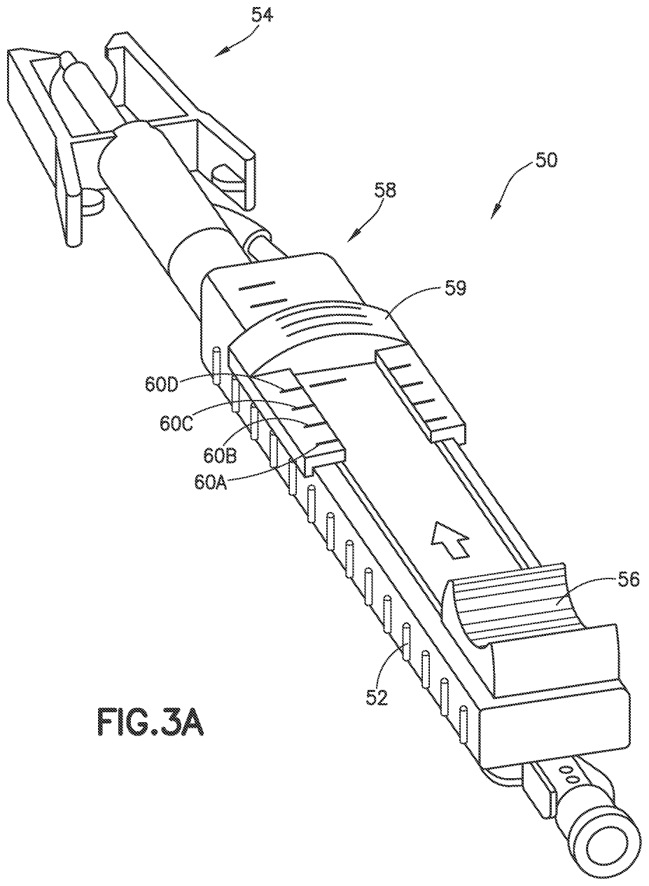

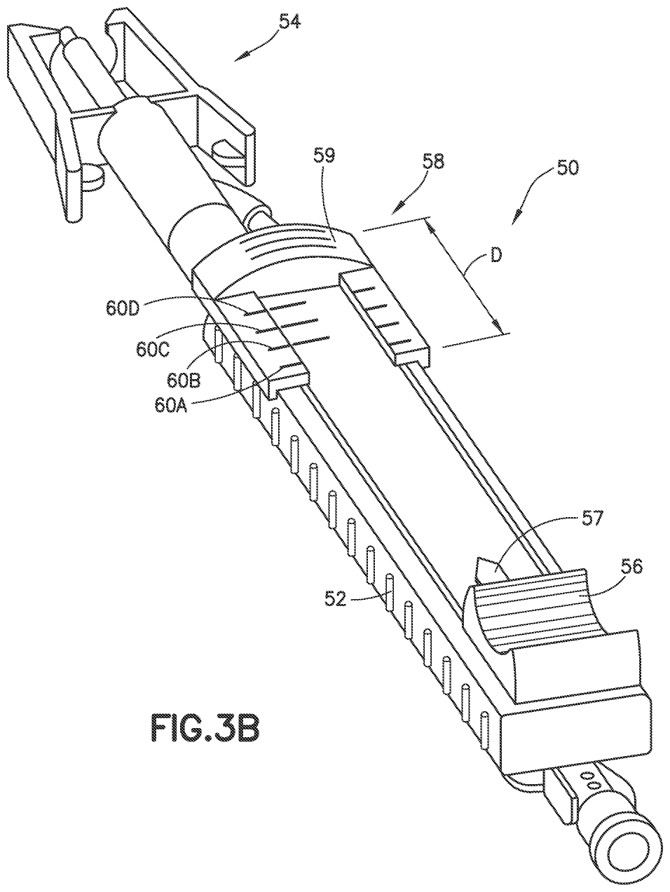

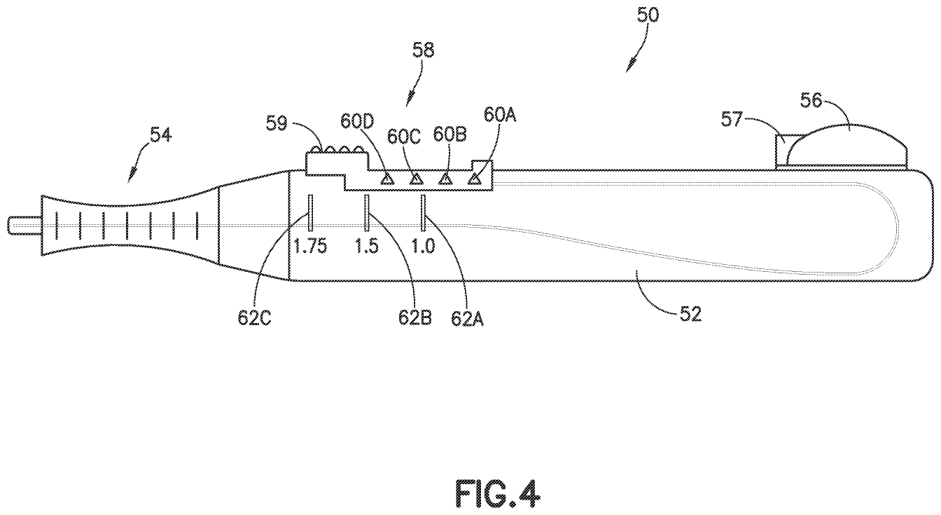

Next, referring to FIGS. 3A-4, a blood draw device 50 in accordance with another aspect of the present disclosure is shown. While not shown in FIGS. 3A-4, it is to be understood that blood draw device 50 is configured for releasable engagement with a catheter system such as that shown and described above with respect to FIGS. 1 and 2.

The blood draw device 50 includes an elongated tube or probe (not shown) that can be selectively advanced in a distal direction beyond the distal tip of an indwelling catheter to which the blood draw device 50 is coupled. The probe of the blood draw device 50 provides a fluid channel to allow for blood draw into a blood collection device coupled to the blood draw device 50. Advancement of the probe of blood draw device 50 is controlled by an actuator 56 linearly movable along a housing 52, which enables a user to push the actuator 56 in a distal direction to advance the probe and, conversely, in a proximal direction to retract the probe. Prior to deployment of the probe, a connector portion 54 of the blood draw device 50 is coupled to, e.g., an adapter of a catheter system. Once secured, the probe may be advanced from the blood draw device 50 by way of linear displacement of the actuator 56.

As detailed above, the indwelling catheter of a catheter system may have various lengths and/or gauges, with the specific length and/or gauge being selected based on, e.g., patient anatomy, application, etc. For example, in some embodiments, the catheter may have lengths of 0.5 inches, 0.75 inches, 1.0 inch, 1.25 inches, 1.5 inches, or 1.75 inches, and may have varying gauges such as, e.g., 18 G, 20 G, 22 G, or 24 G.

The blood draw device 50 also includes an advancement stop member 58. The advancement stop member 58 is linearly displaceable along the housing 52 of blood draw device 50. In some embodiments, the linear displacement of the advancement stop member 58 is limited to a predetermined distal portion of the housing 52. The advancement stop member 58 includes a distal interface portion 59, which provides the user with an interface for selectively advancing or retracting the advancement stop member 58. In some embodiments, an upper surface of the distal interface portion 59 may include ribs or other textured surface features so as to improve the user's ability to manipulate the advancement stop member 58 along the housing 52.

While not shown in FIGS. 3A-4, similar to blood draw device 20 described above, an upper surface of the housing 52 may include a plurality of catheter length indicators. In some embodiments, the catheter length indicators may be projections or detents formed on or in the housing 52 so as to provide discrete stop positions for the advancement stop member 58, and the linear travel of the actuator may be limited to be between the distal-most catheter length indicator and the proximal-most catheter length indicator. Each of the catheter length indicators may refer to a catheter length usable in conjunction with the blood draw device 50. For example, the catheter length indicator may demarcate a position for a 1.75 inch catheter, a position for a 1.5 inch catheter, and a position for a 1.00 inch catheter. However, it is to be understood that the blood draw device 50 is not limited to these catheter lengths, and more or fewer catheter length indicators may be provided on the housing 52.

Before distally advancing the actuator 56 to correspondingly advance the probe from the blood draw device 50, the user first sets the advancement stop member 58 at an appropriate position relative to a known catheter length. For example, referring to FIG. 3A, the advancement stop member 58 is positioned at first catheter length indicator located proximally away from a distal end of the housing 52, which relates to a shorter catheter length (e.g., a 1.0 inch catheter), thereby restricting linear travel of the actuator 56. FIG. 3B, on the other hand, illustrates the advancement stop member 58 being positioned at a second catheter length indicator near the distal end of the housing 52, which relates to a longer catheter length (e.g., a 1.75 inch catheter) and allows for increased linear travel of the actuator 56 as compared to the configuration shown in FIG. 3A. Depending on the position of advancement stop member 58, linear travel of the actuator 56 along the housing 52 is limited, thereby also limiting the linear advancement of the probe from the blood draw device 50.

Referring still to FIGS. 3A-4, in some embodiments, the advancement stop member 58 further includes a probe position indicator portion having laterally opposing portions coupled to, and extending proximally from, the distal interface portion 59, with the laterally opposing portions along extending along opposite sides of the housing 52. The probe position indicator portion is configured to allow substantially unencumbered linear movement of the actuator 56 therethrough over a distance D, providing probe position indicators 60A-60D on at least one top surface thereof.

In some embodiments, the probe position indicators 60A-60D are indicators related to the position of the distal tip of the probe beyond a distal tip of the catheter (not shown) based on the position of the actuator 56. For example, as shown in FIG. 4, the probe position indicator 60A provides an indication as to when the distal tip of the probe is positioned at (or substantially near) the distal tip of the catheter, the probe position indicator 60B provides an indication as to when the distal tip of the probe is positioned approximately 1 cm beyond the distal tip of the catheter, the probe position indicator 60C provides an indication as to when the distal tip of the probe is positioned approximately 2 cm beyond the distal tip of the catheter, and the probe position indicator 60D provides an indication as to when the distal tip of the probe is positioned approximately 3 cm beyond the distal tip of the catheter. The actuator 56 may be provided with an alignment member 57 so as to align the actuator 56 relative to the advancement stop member 58, thereby providing accurate positioning of the actuator 56 and, thus, desired extension of the probe beyond the indwelling catheter.

Utilizing the advancement stop member 58 and probe position indicators 60A-60D, the user is able to verify the advanced position of the probe beyond the distal tip of the indwelling catheter, helping to avoid overextension (or underextension) of the probe beyond the distal tip of the indwelling catheter. Furthermore, because the probe position indicator portion part of the advancement stop member 58 and provides visual indication to the user from a top surface of the blood draw device 50, the distal advancement of the probe is able to be accurately determined and monitored, regardless of the length of the catheter.

In some embodiments, the probe position indicators 60A-60D are solely visual indicators, providing the user with a visual confirmation as to the advancement distance of the probe beyond the catheter. However, in other embodiments, the probe position indicators 60A-60D may provide tactile and/or audible feedback to the user related to the various distal positions of the probe. Additionally and/or alternatively, while four probe position indicators 60A-60D are shown and described with respect to FIGS. 3A-4, it is to be understood that more or fewer probe position indicators may be utilized in accordance with the present disclosure.

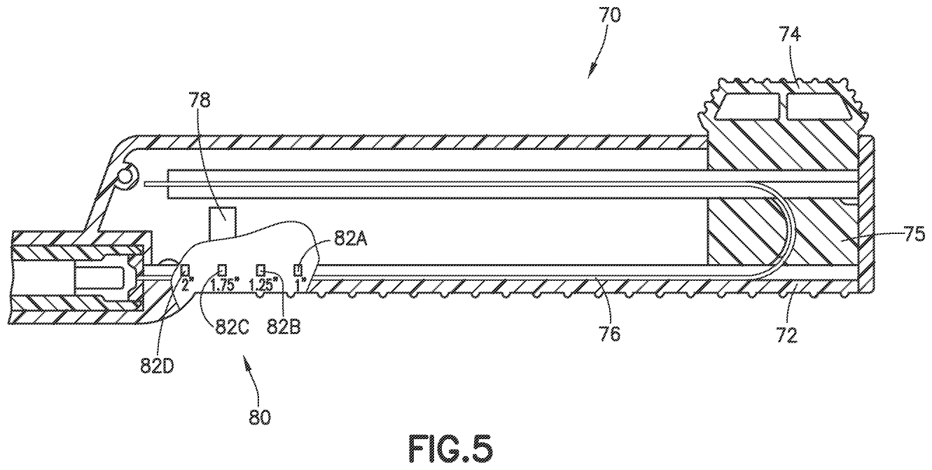

Next, referring to FIG. 5, a blood draw device 70 in accordance with another aspect of the present disclosure is illustrated. Unlike blood draw devices 20 and 50 described above, which include an advancement stop member that is located on an external surface of the introducer body, blood draw device 70 includes an advancement stop member located internal to the introducer body, as will be described further herein.

The blood draw device 70 includes an introducer body 72 having a connector and a blunt introducer (not shown) positioned at a distal end portion. Blood draw device 70 further includes an elongated probe 76 (e.g., a nickel titanium wire, guidewire, instrument, obturator, rod, wire with fluid path and/or sensor, etc.) that can be selectively advanced from the blood draw device in a distal direction, with the probe 76 providing a fluid channel to allow for blood draw into a blood collection device coupled to the blood draw device 70. Advancement of the probe 76 is controlled by an actuator 74, with the probe 76 being operably coupled to an interior portion 75 of the actuator 74 that is linearly slidable along a slot formed in or on the introducer body 72.

Referring still to FIG. 5, the blood draw device 70 further includes an advancement stop member 78, with advancement stop member 78 being positioned internally within the introducer body 72. Similar to actuator 74, advancement stop member 78 is linearly displaceable along at least a portion of the introducer body 72. While not shown in FIG. 5, the advancement stop member 72 may include an external interface portion extending outside of the introducer body 72, which provides the user with an interface for selectively advancing or retracting the advancement stop member 78.

On a lower surface of the introducer body 72 near a distal end portion of the blood draw device 70, a catheter length indicator portion 80 is provided. The catheter length indicator portion 80 includes a plurality of catheter length indicators 82A-82D. In some embodiments, the catheter length indicators 82A-82D may include projections or detents formed on or in the introducer body 72 so as to provide discrete stop positions for the advancement stop member 78. Each of the catheter length indicators 82A-82D refer to a catheter length usable in conjunction with the blood draw device 70. In the example shown in FIG. 5, catheter length indicator 82A demarcates a position for a 1.0 inch catheter, catheter length indicator 82B demarcates a position for a 1.25 inch catheter, catheter length indicator 82C demarcates a position for a 1.75 inch catheter, and catheter length indicator 82D demarcates a position for a 2.0 inch catheter. However, it is to be understood that the blood draw device 70 is not limited to use with these catheter lengths, and more or fewer catheter length indicators may be provided on the introducer body 72. Furthermore, while the example shown in FIG. 5 provides catheter length indicators 82A-82D as being numerical lengths of the respective catheters, it is to be understood that other indicia may be provided such as, e.g., color-coded indicia, etc.

Prior to the user distally advancing the probe 76 from the blood draw device 70 via advancement of the actuator 74, the user first sets the advancement stop member 78 at an appropriate position relative to a known catheter length. For example, in the embodiment shown in FIG. 5, the advancement stop member 78 is positioned at catheter length indicator 82C, which corresponds to a 1.75 inch catheter length. Depending on the position of advancement stop member 78, linear travel of the actuator 74 (and the interior portion 75) along the introducer body 72 is limited, thereby also limiting the linear advancement of the probe 76 from the blood draw device 70.

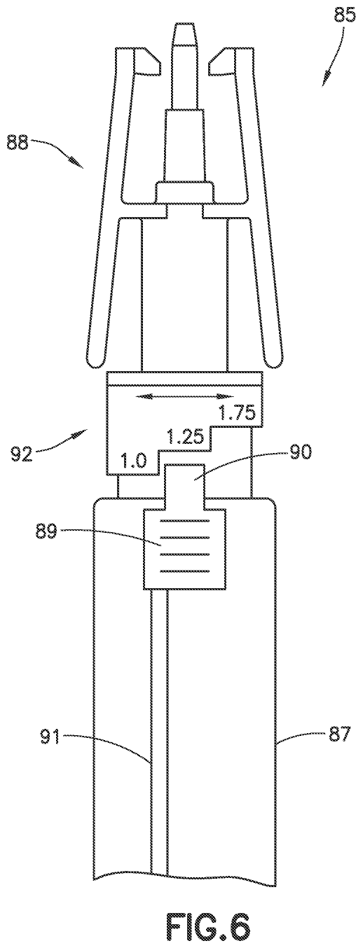

Referring now to FIG. 6, a blood draw device 85 in accordance with another aspect of the present disclosure is illustrated. As will be described in further detail below, blood draw device 85 includes a rotatable advancement stop member in order to selectively limit the linear travel of an actuator and, thus, the linear travel of a flexible probe.

The blood draw device 85 includes an introducer body 87 having a connector and a blunt introducer portion 88 positioned at a distal end thereof. Blood draw device 85 further includes an elongated probe (not shown) (e.g., a nickel titanium wire) that can be selectively advanced from the blood draw device 85 in a distal direction, with the probe providing a fluid channel to allow for blood draw into a blood collection device coupled to the blood draw device 85. Advancement of the probe is controlled by an actuator 89, with the probe being operably coupled to actuator 89 that is linearly slidable along a slot 91 formed in or on the introducer body 72.

Referring still to FIG. 6, the blood draw device 85 further includes a rotatable advancement stop member 92 positioned at a distal end portion the introducer body 72. The advancement stop member 92 includes a plurality of stepped portions, with each stepped portion corresponding to a respective catheter length and having associated catheter length indicators provided thereon. In some embodiments, advancement stop member 92 and/or the introducer body 87 may include projections or detents to provide discrete stop positions when rotating the advancement stop member 92.

Each of the catheter length indicators and stepped portions of the advancement stop member 92 refer to a catheter length usable in conjunction with the blood draw device 85. In the example shown in FIG. 6, catheter length indicators and stepped portions demarcating a position for a 1.0 inch catheter, a 1.25 inch catheter, and a 1.75 inch catheter are provided. However, it is to be understood that the blood draw device 85 is not limited to use with these catheter lengths, and more or fewer stepped portions and/or catheter length indicators may be provided on the advancement stop member 85.

Before the user distally advances the probe from the blood draw device 85 via advancement of the actuator 89, the user first rotates the advancement stop member 92 to set the advancement stop member 92 at an appropriate position relative to a known catheter length. For example, in the embodiment shown in FIG. 6, the advancement stop member 92 rotated to a position corresponding to a 1.25 inch catheter length. Depending on the rotated position of advancement stop member 92, linear travel of the actuator 89 along the introducer body 87 is limited, as a distal extension 90 of the actuator 89 contacts the set stepped portion of the advancement stop member 92 as it is moved to a distal termination position on the introducer body 87. Such limiting of the linear travel of the actuator 89 based on rotation position of the advancement stop member 92 also acts to limit the linear advancement of the probe from the blood draw device 85.

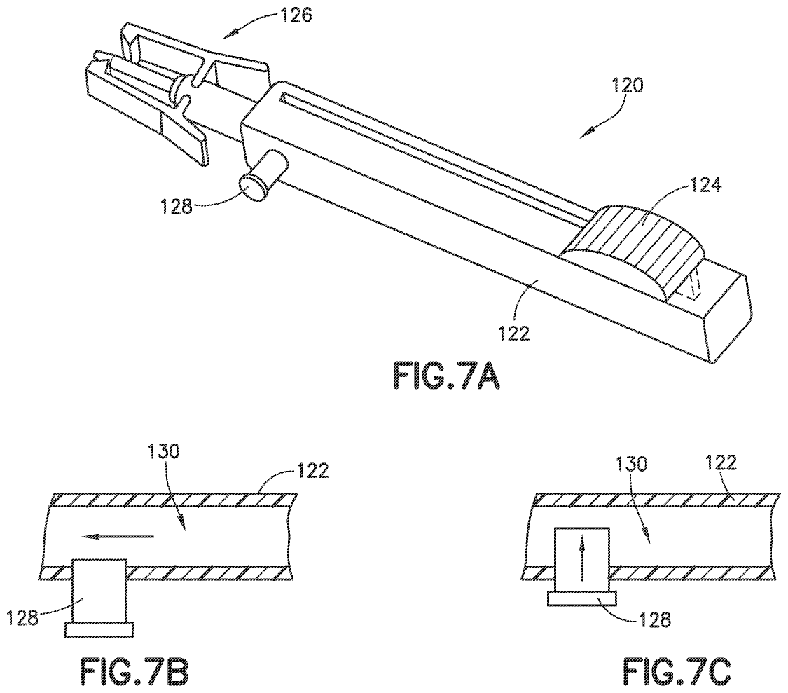

Next, referring to FIGS. 7A-7C, a blood draw device 120 in accordance with another aspect of the present disclosure is illustrated. The blood draw device 120 includes an introducer body 122 having a connector and a blunt introducer portion 126 positioned at a distal end portion thereof. Blood draw device 120 further includes an elongated probe (not shown) (e.g., a nickel titanium wire) that can be selectively advanced from the blood draw device in a distal direction, with the probe providing a fluid channel to allow for blood draw into a blood collection device coupled to the blood draw device 120. Advancement of the probe is controlled by an actuator 124, with the probe being operably coupled to an interior portion of the actuator 124 that is linearly slidable along a slot formed in or on the introducer body 122.

Near a distal end portion of the introducer body 122, the blood draw device 120 further includes an advancement stop member 128, with advancement stop member 128 being positioned on a lateral side of introducer body 128. Advancement stop member 128 is configured to be selectively actuated by the user in a direction substantially transverse to the travel direction of the actuator 124. In some embodiments, the advancement stop member 128 may be configured as a click-type, spring-biased button to enable selective activation and deactivation of the advancement stop member 128.

Prior to the user distally advancing the probe from the blood draw device 120 via advancement of the actuator 124, the user first determines the length of the catheter to which the blood draw device 120 is to be coupled. In some embodiments, when the catheter is a first length (e.g., 1.75 inches), the advancement stop member 128 is not actuated, thereby allowing the actuator 124 unrestricted travel through an interior channel 130 of the introducer body 122, as is shown in FIG. 7B. Such unrestricted travel of the actuator 124 allows full extension of the probe a predetermined distance beyond the distal tip of the catheter, with the length of the probe being optimized based on the longest applicable catheter (e.g., 1.75 inches). However, referring to FIG. 7C, if the catheter is determined to be shorter (e.g., 1.0 inch), the user may actuate the advancement stop member 128 such that a portion of the interior channel 130 is blocked, thereby restricting linear travel of the actuator 124 and, thus, restricting linear travel of the probe.

While FIGS. 7A-7C illustrate only one advancement stop member 128, it is to be understood that two or more advancement stop members may be provided along a lateral side of the introducer body 122, with each advancement stop member 128 correlating to a potential catheter length. Numerical and/or color-coded indicia may be provided for each advancement stop member 128 such that the user can accurately set the advancement stop member 128 prior to a blood draw.

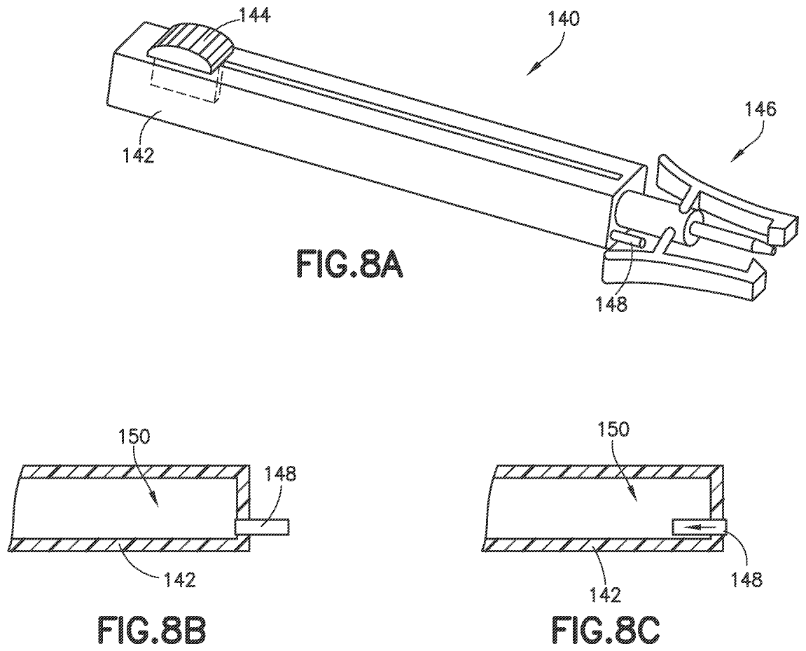

Referring to FIGS. 8A-8C, a blood draw device 140 in accordance with another aspect of the present disclosure is illustrated. The blood draw device 140 includes an introducer body 142 having a connector and a blunt introducer portion 146 positioned at a distal end portion thereof. Blood draw device 140 further includes an elongated probe (not shown) (e.g., a nickel titanium wire) that can be selectively advanced from the blood draw device in a distal direction, with the probe providing a fluid channel to allow for blood draw into a blood collection device coupled to the blood draw device 140. Advancement of the probe is controlled by an actuator 144, with the probe being operably coupled to an interior portion of the actuator 144 that is linearly slidable along a slot formed in or on the introducer body 142.

The blood draw device 140 further includes an advancement stop member 148, with advancement stop member 148 extending from a distal end of the introducer body 142. Advancement stop member 148 is configured to be selectively actuated by the user in a direction substantially parallel to the travel direction of the actuator 144. In some embodiments, the advancement stop member 148 may be configured as a click-type, spring-biased button to enable selective activation and deactivation of the advancement stop member 148.

Prior to the user distally advancing the probe from the blood draw device 140 via advancement of the actuator 144, the user first determines the length of the catheter to which the blood draw device 140 is to be coupled. In some embodiments, when the catheter is a first length (e.g., 1.75 inches), the advancement stop member 148 is not actuated, thereby allowing the actuator 144 unrestricted travel through an interior channel 150 of the introducer body 142, as is shown in FIG. 8B. Such unrestricted travel of the actuator 144 allows full extension of the probe a predetermined distance beyond the distal tip of the catheter, with the length of the probe being optimized based on the longest applicable catheter (e.g., 1.75 inches). However, referring to FIG. 8C, if the catheter is determined to be shorter (e.g., 1.0 inch), the user may actuate the advancement stop member 148 such that a portion of the interior channel 150 is blocked, thereby restricting linear travel of the actuator 144 and, thus, restricting linear travel of the probe.

While FIGS. 8A-8C illustrate only one advancement stop member 148, it is to be understood that two or more advancement stop members may be provided at different locations on the distal end of the introducer body 142, with each advancement stop member 148 correlating to a potential catheter length. Numerical and/or color-coded indicia may be provided for each advancement stop member 148 such that the user can accurately set the advancement stop member 148 prior to a blood draw. Alternatively, the single advancement stop member 148 may be configured to have multiple position settings. For example, in addition to the fully-deactivated position (shown in FIG. 8B) and the fully-activated position (shown in FIG. 8C), the advancement stop member 148 may have one or more intermediate settings to provide an actuator stop for use with catheters having lengths of, e.g., 1.25 inches, 1.5 inches, etc.

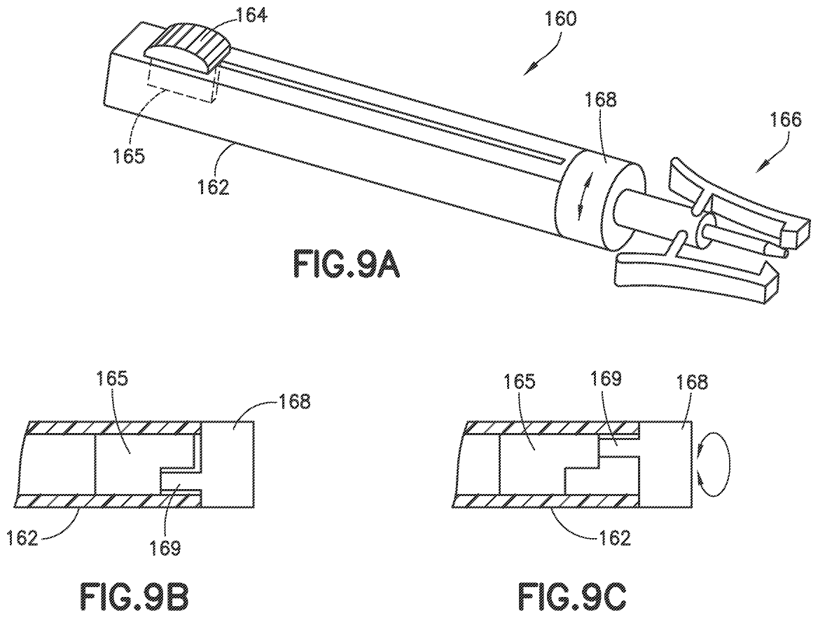

Referring to FIGS. 9A-9C, a blood draw device 160 in accordance with another aspect of the present disclosure is illustrated. The blood draw device 160 includes an introducer body 162 having a connector and a blunt introducer portion 166 positioned at a distal end portion thereof. Blood draw device 160 further includes an elongated probe (not shown) (e.g., a nickel titanium wire) that can be selectively advanced from the blood draw device in a distal direction, with the probe providing a fluid channel to allow for blood draw into a blood collection device coupled to the blood draw device 160. Advancement of the probe is controlled by an actuator 164, with the probe being operably coupled to an interior portion 165 of the actuator 164 that is linearly slidable along a slot formed in or on the introducer body 162.

The blood draw device 160 further includes an advancement stop member 168, with advancement stop member 168 being a rotatable, dial-type actuator positioned on a distal end of the introducer body 162. Advancement stop member 168 is configured to be selectively rotated by the user based on a known catheter length. That is, before distally advancing the probe from the blood draw device 160 via advancement of the actuator 164, the user first determines the length of the catheter to which the blood draw device 160 is to be coupled. In some embodiments, when the catheter is a first length (e.g., 1.75 inches), the advancement stop member 168 is rotated to a first position shown in FIG. 9B, which allows the interior portion 165 of the actuator 164 to travel to a distal end of the introducer body 162. More specifically, the interior portion 165 of actuator 164 may be shaped such that when the advancement stop member 168 is rotated to the first position, the interior portion 165 does not contact a projection 169 extending proximally from the advancement stop member 168. Substantially unrestricted travel of the actuator 164 allows full extension of the probe a predetermined distance beyond the distal tip of the catheter, with the length of the probe being optimized based on the longest applicable catheter (e.g., 1.75 inches).

However, referring to FIG. 9C, if the catheter is determined to be shorter (e.g., 1.0 inch), the user may rotate the advancement stop member 168 such that the projection 169 limits linear travel of the interior portion 165 of actuator 164, thereby also restricting linear travel of the probe.

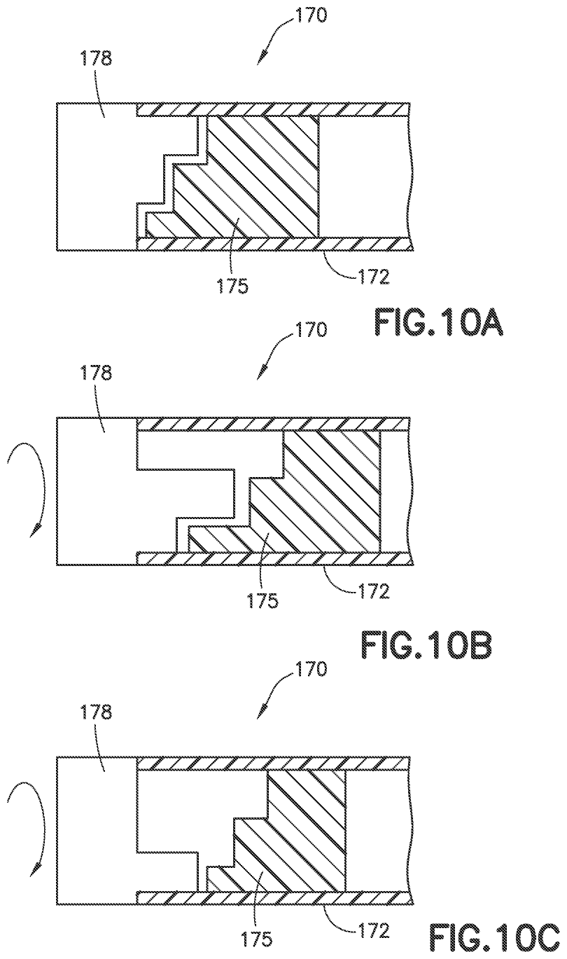

While FIGS. 9A-9C pertain to an advancement stop member 168 configured for only two settings, it is to be understood that a rotatable advancement stop member having more than two settings may be provided. FIGS. 10A-10C illustrate a blood draw device 170 having such a configuration. First, referring to FIG. 10A, a rotatable advancement stop member 178 is provided on a distal end of a introducer body 172, with the advancement stop member 178 having a stepped interior profile extending proximally into the introducer body 172. An interior portion 175 of an actuator (not shown) may have a correspondingly stepped distal profile such that when the advancement stop member 178 is in a first position (shown in FIG. 10A), the interior portion 175 and actuator are capable of full linear travel along the introducer body 172. This first position may correspond to a longer catheter length (e.g., 1.75 inches).

However, if the catheter is determined to be of intermediate length (e.g., 1.25 inches, 1.5 inches, etc.), the advancement stop member 178 may be rotated to a second position, as is shown in FIG. 10B. In this second position, the stepped proximally-directed profile of the advancement stop member 178 may interact with the interior portion 175 to prevent full linear travel of both the actuator and the attached probe. Finally, if the catheter is determined to be an even shorter length (e.g., 1.0 inch), the advancement stop member 178 may be rotated to a third position, as is shown in FIG. 10C, thereby further restricting linear travel of the actuator and the probe. Numerical and/or color-coded indicia may be provided on one or both of the advancement stop member 178 and the introducer body 172 such that the user can accurately set the advancement stop member 178 prior to a blood draw.

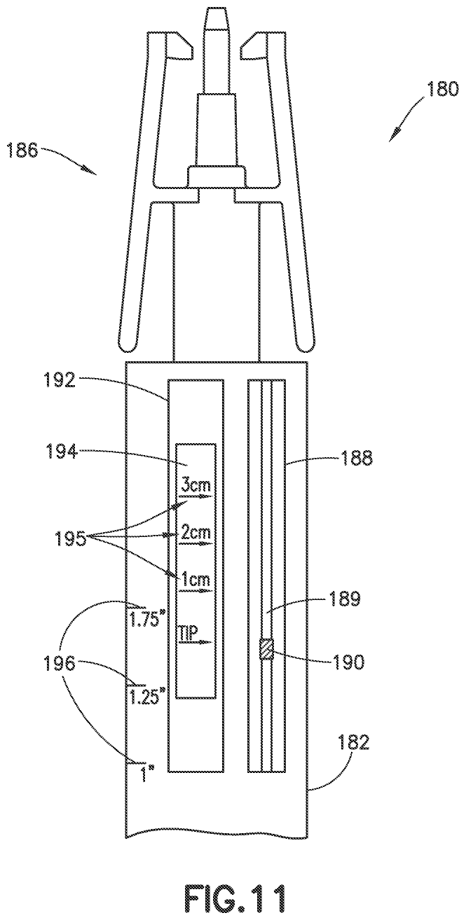

Referring to FIG. 11, a blood draw device 180 in accordance with another aspect of the present disclosure is shown. The blood draw device 180 includes an introducer body 182 having a connector and a blunt introducer portion 186 positioned at a distal end thereof. The blood draw device 180 includes an elongated probe 189 (e.g., a nickel titanium wire) that can be selectively advanced in a distal direction from the blood draw device 180, with the probe 189 providing a fluid channel to allow for blood draw into a blood collection device coupled to the blood draw device 180. Advancement of the probe 189 of blood draw device 180 is controlled by an actuator (not shown) operably coupled to the probe and linearly slidable along a slot formed in or on the introducer body 182, which enables a clinician to use his or her thumb to push the actuator in a distal direction to advance the probe and, conversely, in a proximal direction to retract the probe.

Referring still to FIG. 11, the blood draw device 180 further includes an advancement stop member 194, with advancement stop member 194 being linearly displaceable along a slot 192 formed in the introducer body 182. An upper surface of the introducer body 182 adjacent to the slot 192 is provided with a plurality of catheter length indicators 196. The catheter length indicators 196 may only be visual markings provided on the introducer body 182. Alternatively, in some embodiments, projections or detents may be formed on or in the introducer body 182 so as to provide discrete stop positions for the advancement stop member 194 relative to the catheter length indicators 196. Each of the catheter length indicators 196 refer to a catheter length usable in conjunction with the blood draw device 180. In the example shown in FIG. 11, catheter length indicator 196 demarcates positions for a 1.75 inch catheter, a 1.25 inch catheter, and a 1.00 inch catheter, respectively. However, it is to be understood that the blood draw device 180 is not limited to these catheter lengths, and more or fewer catheter length indicators may be provided on the introducer body 182.

Prior to the user distally advancing the probe 189 from the blood draw device 180 via advancement of the actuator, the user first sets the advancement stop member 194 at an appropriate position relative to a known catheter length. For example, in the embodiment shown in FIG. 11, the advancement stop member 194 is positioned at catheter length indicator 196 relating to a 1.25 inch catheter length. In some embodiments, the advancement stop member 194 may form a physical stop to limit linear travel of the actuator along the introducer body 182, thereby also limiting the linear advancement of the probe 189.

In some embodiments, the advancement stop member 194 further includes a probe position indicators 195. The probe position indicators 195 are indicators related to the position of the distal tip of the probe 189 beyond the distal tip of the catheter based on the position of the actuator. The probe 189 may be provided with a marker 190, which is visible to the user through, e.g., a window 188 formed in a top surface of the introducer body 182. When laterally aligned with the marker 190, the respective probe position indicators 195 provide an indication as to when the distal tip of the probe 189 is positioned at (or substantially near) the distal tip of the catheter, approximately 1 cm beyond the distal tip of the catheter, approximately 2 cm beyond the distal tip of the catheter, and approximately 3 cm beyond the distal tip of the catheter. It is to be understood that more or fewer probe position indicators may be provided on the advancement stop member 194. In this way, the user is able to verify the advanced position of the probe 189 beyond the distal tip of the indwelling catheter, helping to avoid overextension (or underextension) of the probe 189. Furthermore, because the probe position indicators 195 are part of the advancement stop member 194, the distal advancement of the probe 189 is able to be accurately determined and monitored, regardless of the length of the catheter.

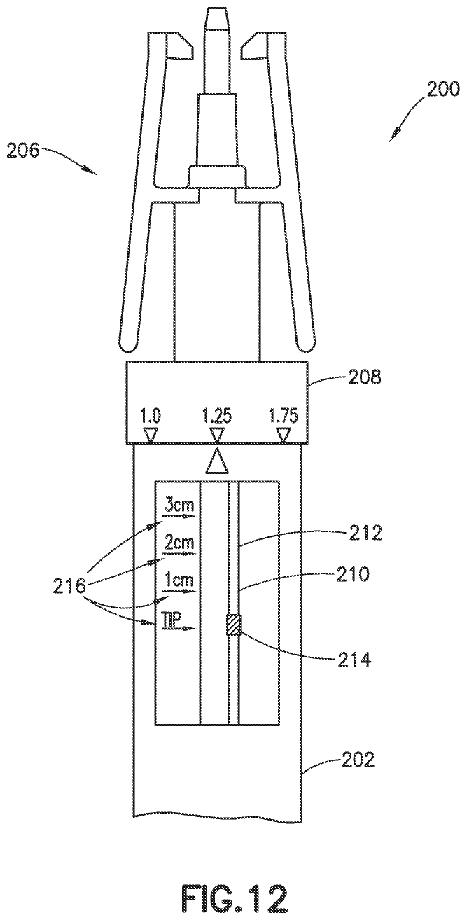

Next, with reference to FIG. 12, a blood draw device 200 in accordance with another aspect of the present disclosure is shown. The blood draw device 200 includes an introducer body 202 having a connector and a blunt introducer portion 206 positioned at a distal end thereof. The blood draw device 200 includes an elongated probe 212 (e.g., a nickel titanium wire) that can be selectively advanced in a distal direction from the blood draw device 200, with the probe 212 providing a fluid channel to allow for blood draw into a blood collection device coupled to the blood draw device 200. Advancement of the probe 212 of blood draw device 200 is controlled by an actuator (not shown) operably coupled to the probe and linearly slidable along a slot formed in or on the introducer body 202, which enables a clinician to use his or her thumb to push the actuator in a distal direction to advance the probe and, conversely, in a proximal direction to retract the probe.

The blood draw device 200 further includes an advancement stop member 208, with advancement stop member 208 being rotatable about a distal end of the introducer body 202. The rotatable advancement stop member 208 is provided with a plurality of catheter length indicators, which refer to a catheter length usable in conjunction with the blood draw device 200. In the example shown in FIG. 12, catheter length indicators demarcates positions for a 1.75 inch catheter, a 1.25 inch catheter, and a 1.00 inch catheter, respectively. However, it is to be understood that the blood draw device 200 is not limited to these catheter lengths, and more or fewer catheter length indicators may be provided on the introducer body advancement stop member 208.

Prior to the user distally advancing the probe 212 from the blood draw device 200 via advancement of the actuator, the user first rotates the advancement stop member 208 to an appropriate setting relative to a known catheter length. For example, in the embodiment shown in FIG. 12, the advancement stop member 208 is rotated to a catheter length indicator relating to a 1.25 inch catheter length. While not shown, it is to be understood that the advancement stop member 208 may include a proximally-directed stepped portion extending within the introducer body 202 similar to those shown and described above with respect to FIGS. 10A-10C, which may form a physical stop to limit linear travel of the actuator along the introducer body 202, thereby also limiting the linear advancement of the probe 212.

In some embodiments, the introducer body 202 further includes a plurality of probe position indicators 216. The probe position indicators 216 are indicators related to the position of the distal tip of the probe 212 beyond the distal tip of the catheter based on the position of the actuator. The probe 212 may be provided with a marker 214, which is visible to the user through, e.g., a window 210 formed in a top surface of the introducer body 202. When laterally aligned with the marker 210, the respective probe position indicators 216 provide an indication as to when the distal tip of the probe 212 is positioned at (or substantially near), e.g., the distal tip of the catheter, approximately 1 cm beyond the distal tip of the catheter, approximately 2 cm beyond the distal tip of the catheter, and approximately 3 cm beyond the distal tip of the catheter. However, it is to be understood that more or fewer probe position indicators 216 may be provided on the introducer body 202. In this way, the user is able to verify the advanced position of the probe 212 beyond the distal tip of the indwelling catheter, helping to avoid overextension (or underextension) of the probe 212. Furthermore, because the linear travel of the actuator (and, thus, the probe 212) may be limited by the advancement stop member 208, the distal advancement of the probe 212 is able to be accurately determined and monitored, regardless of the length of the catheter.

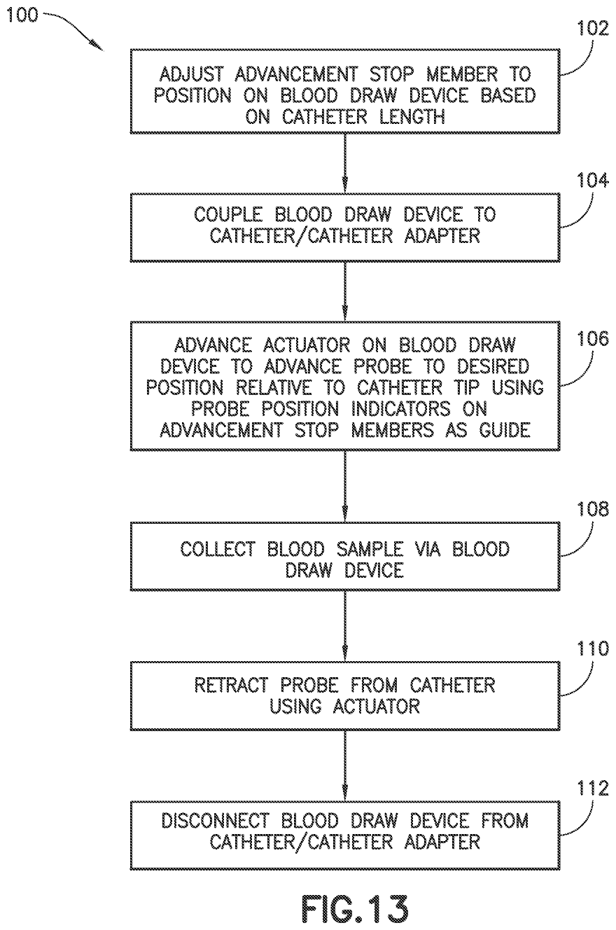

Referring now to FIG. 13, a method 100 of using a blood draw device in accordance with an aspect of the present disclosure is illustrated. First, at 102, an advancement stop member on the blood draw device is adjusted into an appropriate position based on a known length of a catheter. After the advancement stop member is positioned, the blood draw device is coupled to the catheter (or catheter adapter) at 104. Next, at 106 the actuator on the blood draw device is linearly advanced so as to correspondingly advance the flexible probe to a desired position relative to (and beyond) a distal tip of the catheter, with probe position indicator provided on the advancement stop member used as a guide to determine the distal position of the probe tip relative to a distal tip of the catheter.

Once the probe is positioned at a desired location relative to the catheter tip, at 108, a blood sample is collected via that blood draw device. Then, at 110, the probe is retracted from the catheter using the actuator. Finally, at 112, the blood draw device is disconnected from the catheter/catheter adapter. In some embodiments, the blood draw device is a single-use device and may be disposed of in an appropriate container after use.

While several embodiments of blood draw devices configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A blood draw device for delivery of a probe into a patient's vascular system, the blood draw device comprising:
an introducer body having a proximal end and a distal end;
an actuator, wherein the actuator is displaceable along at least a portion of the introducer body, and wherein the actuator is operably coupled to the probe to advance and retract the probe based on direction of displacement of the actuator; and
an advancement stop member, wherein the advancement stop member is displaceable along at least a portion of the introducer body and is configured to selectively limit travel of the actuator along the introducer body.

2. The blood draw device of claim 1, wherein the advancement stop member comprises a distal interface portion and a probe position indicator portion, wherein the probe position indicator portion is coupled to and extends proximally from the distal interface portion.

3. The blood draw device of claim 2, wherein the probe position indicator portion extends along at least one sidewall of the introducer body.

4. The blood draw device of claim 2, wherein the probe position indicator portion extends along two opposing sides of the introducer body.

5. The blood draw device of claim 2, wherein the probe position indicator portion comprises a plurality of probe position indicators, wherein each of the probe position indicators relate to a position of a distal tip of the probe relative to a distal tip of an indwelling catheter to which the blood draw device is coupled.

6. The blood draw device of claim 5, wherein the plurality of probe position indicators comprise visual indicators.

7. The blood draw device of claim 6, wherein the visual indicators comprise indications of when the distal tip of the probe is a) at the distal tip of the indwelling catheter, b) 1 cm beyond the distal tip of the indwelling catheter, c) 2 cm beyond the distal tip of the indwelling catheter, and d) 3 cm beyond the distal tip of the indwelling catheter.

8. The blood draw device of claim 5, wherein the plurality of probe position indicators are configured to provide tactile feedback to the user.

9. The blood draw device of claim 1, wherein the introducer body comprises a plurality of catheter length indicators formed on a distal end portion thereof.

10. The blood draw device of claim 9, wherein each of the catheter length indicators corresponds to a length of a catheter to which the blood draw device is coupled.

11. The blood draw device of claim 9, wherein the advancement stop member is configured to selectively engage with the catheter length indicators so as to provide discrete stop positions for the advancement stop member along the introducer body.

12. The blood draw device of claim 9, wherein each of the catheter length indicators comprise one of a projection or a detent.

13. The blood draw device of claim 9, wherein each of the catheter length indicators comprise visual indicia pertaining to a catheter length.

14. The blood draw device of claim 1, further comprising a connector and a blunt introducer extending from the distal end of the introducer body.

15. A blood draw device for delivery of a probe into a patient's vascular system, the blood draw device comprising:
an introducer body having a proximal end and a distal end;
an actuator, wherein the actuator is displaceable along at least a portion of the introducer body, and wherein the actuator is operably coupled to the probe to advance and retract the probe based on direction of displacement of the actuator; and
an advancement stop member, wherein the advancement stop member is rotatable about the introducer body and is configured to selectively limit travel of the actuator along the introducer body.

16. The blood draw device of claim 15, wherein the advancement stop member comprises a proximally-directed stepped portion configured to extend into the introducer body.

17. A method of blood collection from a vascular access device, the method comprising:
providing a catheter assembly having a catheter of known length;
providing a blood draw device, the blood draw device comprising:
an introducer body having a proximal end and a distal end,
an actuator, wherein the actuator is displaceable along at least a portion of the introducer body, and wherein the actuator is operably coupled to a flexible probe to advance and retract the probe based on direction of displacement of the actuator, and
an advancement stop member, wherein the advancement stop member is displaceable along at least a portion of the introducer body and is configured to selectively limit travel of the actuator along the introducer body;
adjusting the advancement stop member to a position on the introducer body based on the known length of the catheter;
coupling the blood draw device to the catheter assembly;
advancing the actuator to correspondingly advance the probe from the blood draw device and beyond a distal tip of the catheter; and
collecting a blood sample via the blood draw device.

18. The method of claim 17, wherein the advancement stop member comprises a distal interface portion and a probe position indicator portion, and wherein the probe position indicator portion is coupled to and extends proximally from the distal interface portion.

19. The method of claim 18, wherein the step of advancing the actuator on the blood draw device comprises using the probe position indicator portion as a guide to determine desired travel of the probe beyond the distal tip of the catheter.

20. The method of claim 17, further comprising:
retracting the probe from the catheter after collection of the blood sample using the actuator; and
decoupling the blood draw device from the catheter assembly.
